(19) United States
(12) Patent Application Publication
(10) Pub. No.: US 2015/0151068 A1
(43) Pub. Date: Jun. 4, 2015

(54) PATIENT INTERFACE DEVICE

(71) Applicant: KONINKLIJKE PHILIPS N.V., EINDHOVEN (NL)

(72) Inventors: Christoph Dobrusskin, Eindhoven (NL); Sander Theodoor Pastoor, Utrecht (NL)

(73) Assignee: KONINKLIJKE PHILIPS N.V., EINDHOVEN (NL)

(21) Appl. No.: 14/408,584

(22) PCT Filed: Jun. 13, 2013
(86) PCT No.: PCT/IB2013/054850

§ 371 Date: Dec. 17, 2014

Related U.S. Application Data
(60) Provisional application No. 61/662,940, filed on Jun. 22, 2012.

(51) Int. Cl. A61M 16/06; A61M 16/00

(52) U.S. Cl. CPC A61M 16/0622; A61M 16/0003; A61M 16/0644; A61M 16/0683; A61M 2205/332

(57) ABSTRACT

The present invention relates to a patient interface device (10) comprising a sealing assembly (12) for delivering a flow of breathable gas to a patient (16),an attachment assembly (14) for attaching the sealing assembly (12) to a patient's face (18) and a force limitation unit (26)for limiting a force between the patient interface device(10) and the patient's face (18) to a maximum force when the patient interface device (10) is applied to the patient (16), the force limitation unit (26) comprising a first and a second coupling element (27, 28) constituting a self-releasing force-fitted coupling for transmitting the force from the first to the second coupling element (27, 28) and for interrupting the force-fit between said two coupling elements (27, 28) when the force exceeds the maximum force.

### FIELD OF THE INVENTION

The present invention relates to a patient interface device comprising a sealing assembly for delivering a flow of breathable gas to a patient and an attachment assembly for attaching the sealing assembly to a patient's face.

### BACKGROUND OF THE INVENTION

More and more patients suffer from obstructive sleep apnea or obstructive sleep apnea syndrome (OSA). OSA is usually caused by an obstruction of the upper airway. It is characterized by repetitive pauses in breathing during sleep and is usually associated with a reduction in blood oxygen saturation. These pauses in breathing, called apneas, typically last 20 to 40 seconds. The obstruction of the upper airway is usually caused by reduced muscle tonus of the body that occurs during sleep. The human airway is composed of walls of soft tissue which can collapse and thereby obstruct breathing during sleep. Tongue tissue moves towards the back of the throat during sleep and thereby blocks the air passages. OSA is therefore commonly accompanied with snoring.

Different invasive and non-invasive treatments for OSA are known. One of the most powerful non-invasive treatments is the usage of CPAP (continuous positive airway pressure) or BiPAP (bi-positive airway pressure) in which a facial mask (generally called “patient interface device” hereinafter) is attached to a tube and a machine that blows pressurized air into the mask and through the airway in order to keep it open. Positive air pressure is thus provided to a patient through a hose connected to a patient interface, such as a facial mask, that is worn by the patient. Usually, these facial masks are worn using a head gear with straps that go around the back of the patient's head. An example of such a CPAP system is known from WO 2011/022779 A1.

Patient interface devices and facial masks respectively are used in pressure support systems that supply a flow of gas to an airway of a patient. These systems are well-known, and are mostly worn on the head using a strap system around the head to hold the mask in place on the face, and also using a mechanism with an additional cushion support on the forehead to balance the forces put by the mask around the airway entry features of the human face.

A problem facing the design of these masks is the wide variety of shapes and forms present in the human race, which makes it difficult to design a single mask that fits a large part of the population. Therefore a variety of adjustment mechanisms have been designed to adjust the forces put on the face of the wearer of such a mask, which should be high to provide a seal between the mask part covering the nose and/or mouth part of the human face, and which at the same time should be low to allow for a comfortable fit, avoid red marks and skin damage on the face.

A disadvantage of most of the well-known adjustment mechanisms is the fact that the user of such a mask is adjusting the force that the mask exerts on the face by him or herself. Being generally not an expert in those matters the masks are often either put on with too high a force or too low a force. Too high a force may easily cause discomfort, leave visually very distractive red marks or even cause skin damage and significant pain. Too low a force may cause air leaks, defeating the purpose of the pressure support system, and cause a skin discomfort as well as waking-up sleeping patients due to unpleasant noises. Additionally, the air can also leak into the patient's eyes, causing eye irritation. The above described problems reduce the patient tolerance and compliance with the medical procedures utilizing the mask.

### SUMMARY OF THE INVENTION

It is an object of the present invention to provide a patient interface device that overcomes the shortcomings of the conventional adjustment mechanisms applied by well known facial masks.

According to an aspect of the present invention a patient interface device is provided comprising a sealing assembly for delivering a flow of breathable gas to a patient, an attachment assembly for attaching the sealing assembly to a patient's face and a force limitation unit for limiting a force between the patient interface device and the patient's face to a maximum force when the patient interface device is applied to the patient, wherein the force limitation unit comprises a first and a second coupling element constituting a self-releasing force-fitted coupling for transmitting the force from the first to the second coupling element and for interrupting the force-fit between said two coupling elements when the force exceeds the maximum force.

According to a further aspect of the present invention a force limitation unit is provided for use in a patient interface device.

According to a further aspect of the invention a headgear assembly is provided comprising a force limitation unit.

Preferred embodiments of the invention are defined in the dependent claims. It shall be understood that the claimed force limitation unit has similar and/or identical preferred embodiments as the claimed patient interface device and as defined in the dependent claims.

The patient interface device according to the present invention provides a force limitation unit in at least one of its elements, which are used to attach the mask to the head in an adjustable way. When fixing the patient interface device to the head according to conventional adjustment mechanisms, the adjustment of the force is proceeding from a loose position to a more tight position. According to the present invention, in contrast, the patient interface device is positioned in such a way that a given maximum force cannot be exceeded. In order to achieve a limitation of the force exerted on the patient's face, the force limitation unit features a self-releasing force-fitted coupling that transmits the force from a first to a second coupling element and that interrupts the force-fit between said two coupling elements when the force exceeds the maximum force. This means that the force-fit can be interrupted, for example, for a predetermined temporal or spatial interval in order to make sure that the force exerted on the patient's face does not exceed the maximum force. The force limitation unit assures a proper fit of the patient interface device on the patient's face. Red marks or even open sores resulting from forces on the patient's face being too high are thus avoided. The proposed adjustment mechanism also prevents air leaks causing skin and eye irritations. As a result, the compliance with medical procedures can be significantly increased.

Advantageously, the force limitation unit may be applied regardless whether the force between the patient interface device and the patient's face is provided by the patient or by any other means such as a motor, air pressure and the like. Moreover, it is disclosed that the force limitation unit limits the force when the patient interface device is applied to the patient. This means that the force limitation unit may be employed not only during the fixation of the mask on the face of the patient, but also during use, when, for example, forces are exerted on the mask by the patient sleeping on his or her side or front side. If the force limitation unit provides an automatic adjustment of the force between the patient interface device and the patient's face, the fitting of the mask can be automatically optimized for each situation.

In a first embodiment, the self-releasing force-fitted coupling is adapted to interrupt the force-fit between said two coupling elements by initiating a slip between said two coupling elements when the force exceeds the maximum force.

This means that the force-fit can be interrupted according to a smooth continuous transition from a coupled state to a decoupled state. The smooth transition assures a comfortable fitting of the mask.

In a further embodiment, the self-releasing force-fitted coupling is adapted to interrupt the force-fit between said two coupling elements by at least partly releasing the linking between said two coupling elements when the force exceeds the maximum force.

In other words, the linking between said two coupling elements is released at least until a predetermined displacement between said two coupling elements is reached, thereby implementing a ratchet mechanism. The restriction of the free movement of said two coupling elements can also act as a safety catch in order to prevent the patient interface device from falling off the face of the patient.

In a further embodiment, the self-releasing force-fitted coupling is adapted to interrupt the force-fit between said two coupling elements by completely releasing the linking between said two coupling elements when the force exceeds the maximum force.

In this embodiment, the linking between the coupling elements is completely released at once when the force exceeds the maximum force. This assures that the maximum force exerted on the patient's face cannot be exceeded.

In a further embodiment, the attachment assembly comprises a head gear component coupled to the sealing assembly and comprising the force limitation unit. In this embodiment the force limitation unit is provided, for example, by head gear straps providing a flexible force between the head of the patient and the sealing assembly. The adjustment of the mask is performed by shortening or lengthening the head gear straps. The force limitation unit assures that the maximum force is not exceeded.

In a further embodiment, the attachment assembly comprises a forehead support coupled to the sealing assembly and comprising the force limitation unit. The forehead support is used to provide a varying distance and/or angle between the forehead of the patient and the sealing assembly. The force limitation unit of the forehead support makes sure that a proper angle and distance of the forehead support are set dependent on the individual facial geometry and that excessive forces on the forehead of the patient are avoided. Headaches, for examples, as a result of high pressure on the forehead can be prevented.

In another embodiment, the sealing assembly comprises a rigid mask shell and a cushion coupled to the mask shell, the force limitation unit being mechanically coupled between the mask shell and the cushion. In this embodiment the sealing assembly is pressed to the patient's face, for example, by adjustable head gear straps. The head gear straps provide a flexible force between the head of the patient and the cushion. The force limitation unit between the rigid mask shell and the soft cushion assures that the force exerted on the patient's face does not exceed the maximum force.

According to another embodiment, the force limitation unit comprises setting means for adjusting the maximum force. If the force limitation unit provides setting means for adjusting the maximum force, the maximum force can, for example, be preconfigured during the production process dependent on the mask type and/or mask material. Furthermore, this feature enables the medical staff to adjust the maximum force of the force limitation unit individually during a fitting procedure to take into account individual preferences, characteristics and sensitivities of the patients.

In another embodiment, the patient interface device further comprises a sensor for measuring the force exerted by the patient interface device on the patient's face and/or an actuator or motor for attaching the patient interface device to the patient's face, the setting means being configured to adjust the maximum force based on an output signal of the sensor, actuator and/or motor.

A sensor can be used to measure the forces applied to the patient's face when, for example, shortening the head gear straps. If the forces increase disproportionately high, the maximum force is set, for example, to the actual force value. This, in turn, initiates the slip of the adjustment mechanism. As an alternative, the feedback of an actuator or motor can be used as a sensor for determining the maximum force value. For example, if the electrical current of an electric motor used to adjust the headgear straps increases disproportionately high, the maximum force is determined as the actual force value. As another alternative, a threshold value can be defined for the electrical current that triggers the setting of the maximum force. The determination of the maximum force of the force limitation unit can be done each time when the patient interface is donned by the patient or only once during a specific calibration procedure.

In another embodiment, the patient interface device comprises a plurality of force limitation units being arranged on different locations of the patient interface device. In this embodiment, several force limitation units are applied on different locations of the mask like, for example, the head gear straps, the forehead support, the interface between the mask shell and the cushion and/or any other location of the mask that influences the forces exerted on the patient's face. The usage of a plurality of force limitation units leads to an improved adjustment of the patient interface device.

If each or at least a part of the force limitation units comprises setting means for adjusting the maximum force, the forces applied to different portions of the patients face can be different in order to reflect, for example, different sensitivities of the skin. This leads to a better fitting of the patient interface device and significantly reduces skin and eye irritations. This arrangement is particularly useful to accommodate varying external forces on the mask that may be induced involuntarily.

According to another embodiment, the force limitation unit comprises a friction coupling, a magnetic coupling, a slip coupling, a hydraulic coupling and/or an electromagnetic coupling. The force limitation unit can be implemented by different mechanisms and techniques. However, they all have in common that the force limitation unit features a self-releasing force-fitted coupling for transmitting a force from a first to a second coupling element and for interrupting the force-fit between said two coupling elements when the force exceeds the maximum force.

Even if the force limitation unit is described in the context of a patient interface device, it shall be understood that the force limitation unit can also be applied in any other type of a mask worn by a person. The application of the force limitation unit avoids red marks and skin damage on a person's face and at the same time provides good sealing properties of the patient interface device.

### BRIEF DESCRIPTION OF THE DRAWINGS

These and other aspects of the invention will be apparent from and elucidated with reference to the embodiment(s) described hereinafter. In the following drawings

FIG. 1 shows a first embodiment of the disclosed patient interface device comprising a force limitation unit;

FIG. 2 shows a second embodiment of the disclosed patient interface device; and

FIG. 3 shows a third embodiment of the disclosed patient interface device.

### DETAILED DESCRIPTION OF THE INVENTION

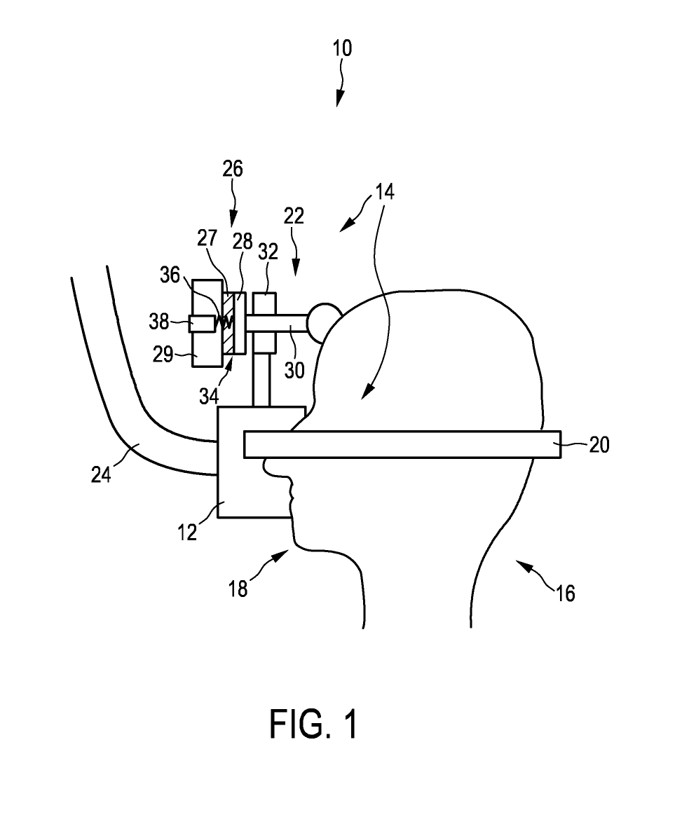

FIG. 1 shows a first embodiment of a patient interface device 10. The patient interface device 10 can, for example, be used in the treatment of Obstructive Sleep Apnea (OSA). The patient interface device 10 comprises a sealing assembly 12 and an attachment assembly 14. The sealing assembly 12 is typically covering the nose and mouth of a patient 16 and delivers a flow of breathable gas to the patient 16. Alternatively, it is also possible that the sealing assembly 12 covers the entire face or only the nose of the patient 16. In another alternative embodiment, the sealing assembly 12 can also comprise cannulas or pillows that are inserted into the nose of the patient 16.

The sealing assembly 12 is attached to a patient's face 18 using the attachment assembly 14. In this embodiment the attachment assembly 14 comprises headgear straps 20 and a forehead support 22. The headgear straps 20 are providing a flexible force between the head of the patient 16 and the sealing assembly 12 and the forehead support 22 respectively.

The forehead support 22 is connected to the sealing assembly 12 and rests against a forehead of the patient 16. In order to accommodate variations in the features of different patients, the forehead support 22 can be adjusted to provide a varying distance and/or angle between the forehead and a plane defined by the sealing assembly 12, the plane being substantially parallel to the patient's face 18.

During use, the sealing assembly 12 is connected via a hose 24 to an air compressor (not shown in FIG. 1) that functions as a source of pressurized air that is used during the medical treatment. The pressurized air is led to the mouth and nose of the patient 16 by means of the sealing assembly 12.

It is a problem of patient interface devices that they have to provide good sealing capabilities around the mouth and nose of the patient 16 and at the same time should provide a comfortable fit. It is a drawback of well known patient interface devices and the respective attachment mechanisms that the masks are often either put on with too high a force or too low a force. If the patient interface is put on too tightly, this results in red marks on a user's face or other forms of discomfort. Too low a force may cause air leaks, defeating the purpose of the pressure support system, and may cause skin and eye irritations.

In order to overcome the above described problem, the patient interface device 10 comprises a force limitation unit 26, which limits the force between the patient interface device 10 and the patient's face 18 to a maximum force when the patient interface device 10 is applied to the patient 16. According to the present invention the force limitation unit 26 comprises a first and a second coupling element 27, 28 constituting a self-releasing force-fitted coupling for transmitting the force from the first to the second coupling element 27, 28 and for interrupting the force-fit between said two coupling elements 27, 28 when the force exceeds the maximum force. In this example, it is assumed that the force-fit is interrupted by initiating a slip between said two coupling elements 27, 28. In alternative embodiments, the force-fit can also be interrupted by releasing the linking between said two coupling elements 27, 28 at least until a predetermined displacement between said two coupling elements 27, 28 is reached. Due to the proposed force limitation unit 26, the force exerted on the patient's face 18 is optimized and red marks or even open sores on the patient's face 18 can be avoided. Additionally, the proposed adjustment mechanism of the patient interface device 10 prevents air leaks causing skin and eye irritations. As a consequence, the patient tolerance and compliance with the medical procedures utilizing the patient interface device 10 can be significantly increased.

In the embodiment of the patient interface device 10 shown in FIG. 1, the force limitation unit 26 is comprised by the forehead support 22. Further, in this embodiment the force limitation unit 26 comprises a knob 29, a screw 30, a nut 32 and a friction coupling 34 constituting the self-releasing force-fitted coupling. The adjustment of the patient interface device 10 is done by turning the knob 29, which moves the screw 30 in the nut 32. The nut 32 has a matching structure to the screw 30 and may be provided with a locking or ratchet mechanism. The knob 29 is coupled to the screw 30 via the friction coupling 34, which limits the force exerted on the patient's face 18 to a maximum force. The friction coupling 34 can comprise, for example, two plates representing the first and second coupling elements 27, 28 and being freely rotatable with respect to each other, but connected by the force of a spring 36. As soon as the actual force exceeds the maximum force value, the two plates slip in respect to each other and the force is limited to the maximum force value.

Furthermore, the force limitation unit 26 comprises setting means 38, in the present example a calibration screw 38, to adjust the maximum force, which is based on the friction between the two plates. In order to adjust the maximum force, the calibration screw 38 acts on the spring 36 and changes its tension and thus the friction between the two plates. By means of the calibration screw 38, the medical staff, for example, can adjust the maximum force value to take into account individual preferences, characteristics and sensitivities of the patient 16.

Even if the friction coupling 34 has been described using the example of two plates, it shall be understood that the friction coupling 34 can be replaced by any other means featuring the above described coupling properties of the self-releasing force-fitted coupling. This includes, for example, magnetic couplings, slip couplings, hydraulic couplings or electromagnetic couplings.

With slip couplings the friction force is provided by a mechanical load between two plates and surfaces respectively. This can be achieved, for example by springs. An alternative way to create the load would be to use magnetic or electromagnetic forces. One embodiment would be to change the preload on the plates by magnetic means (known as electromagnetic clutch). A second embodiment would be to use the magnetic forces directly without the necessity of two plates touching each other in order to provide the coupling. Yet another way to create the load would involve hydraulic forces. In this embodiment pumps and valves would provide control means for such a coupling. The fluids involved in this embodiment could further be modified using electric or magnetic means (e.g. electro-rheological or magneto-rheological fluids).

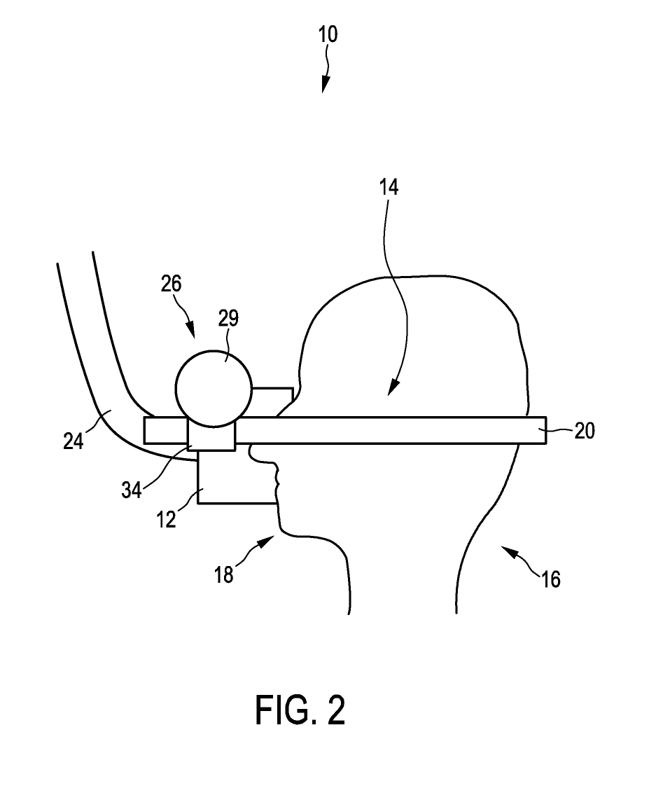

FIG. 2 shows another embodiment of the patient interface device 10. In the following only the differences in respect to FIG. 1 are described. Same reference signs refer to the same elements.

In this embodiment the attachment assembly 14 only comprises the head gear straps 20 for attaching the sealing assembly 12 to the patient's face 18. In an alternative embodiment the attachment assembly 14 can additionally comprise the forehead support 22 as provided in the embodiment shown in FIG. 1.

The headgear straps 20 are providing a flexible force between the head of the patient 16 and the sealing assembly 12 by virtue of elastic properties of the sealing assembly 12, the headgear straps 20 or the mounting of one onto the other. The adjustment of the patient interface device 10 is performed by shortening or lengthening the headgear straps 20. This shortening or lengthening is performed using the knob 29 that can be rotated by the patient 16. The knob 29 is coupled to the headgear straps 20 by means of the friction coupling 34 that limits the maximum force that can be exerted between the headgear straps 20 and the head of the patient 16.

As a further option, the force limitation unit 26 may comprise setting means 38 as provided in the embodiment shown in FIG. 1 to adjust the maximum force of the friction coupling 34.

Due to the proposed force limitation unit 26, the headgear straps 20 can only be pulled tight until the maximum force value is reached. If the actual force exceeds the maximum force value, the force limitation unit 26 gives way, thus limiting the maximum force on the headgear straps 20.

In the embodiments illustrated in the FIGS. 1 and 2 setting means 38 are provided which comprise the calibration screw 38 for a manual adjustment of the maximum force. As an alternative, sensors may be used to automatically determine the maximum force exerted on the patient's face 18. As a result, no manual interaction is needed anymore in order to correctly calibrate the maximum force of the patient interface device 10. The sensors may be integrated in the headgear straps 20 and may be configured to measure the force between the patient interface device 10 and the patient's face 18. As soon as the measured force increases disproportionately high when shortening the headgear straps 20, the actual force is taken as the maximum force and the slip of the adjustment mechanism is initiated.

In a further alternative embodiment the knob 29 illustrated in the FIGS. 1 and 2 and used by the patient 16 to manually adjust the patient interface device 10 to the patient's face 18 can be replaced by an actuator or motor that drives the adjustment mechanism. In this embodiment, for example, an output signal from the actuator or motor may be used as a sensor to determine the maximum force exerted on the patient's face 18. This can be done by monitoring for example the electrical current of an electric motor. As soon as the electrical current increases disproportionately high the actual force is determined as the maximum force and the slip of the adjustment mechanism is initiated.

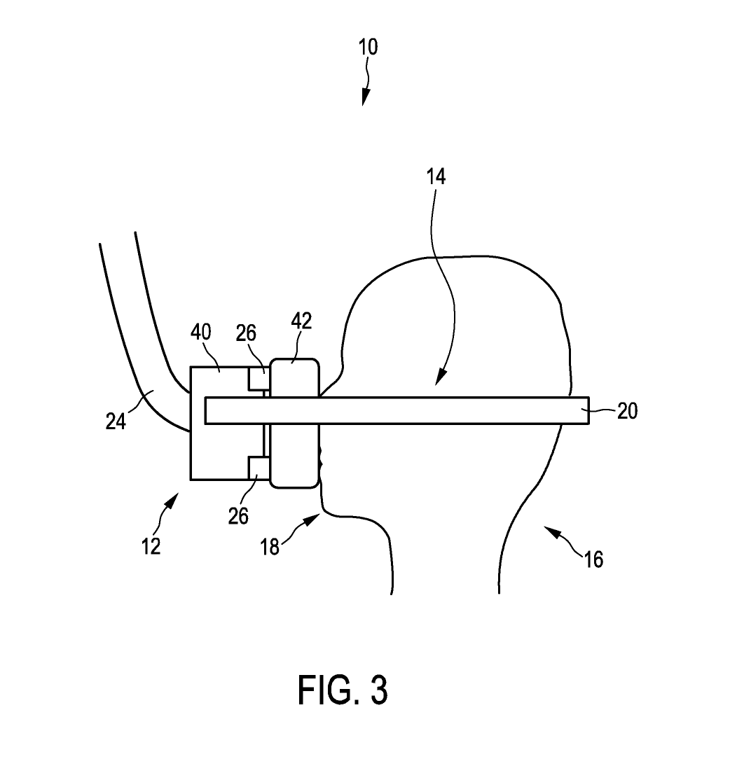

FIG. 3 shows another embodiment of the patient interface device 10. In the following only the differences in respect to FIG. 1 are described. Same reference signs refer to the same elements.

The sealing assembly 12 comprises a rigid mask shell 40 and a cushion 42 that is coupled to the mask shell 40. The mask shell 40 is connected via the hose 24 to an air compressor that functions as a source of pressurized air that is used during the medical treatment. The soft cushion 42 covers the nose and/or mouth of the patient 16 and forms a seal between the patient interface device 10 and the patient's face 18. The mask shell 40 is held to the patient's face 18 by the attachment assembly 14, which comprises the adjustable headgear straps 20. The headgear straps 20 are providing a flexible force between the head of the patient 16 and the cushion 42 by virtue of elastic properties of the mask shell 40, the cushion 42, the head gear straps 20 or the mounting of one onto the other. In order to prevent the sealing force on the patient's face 18 to exceed a maximum force, the cushion 42 is connected to the mask shell 40 by means of one or more force limitation units 26. When the force on the force limitation unit 26 exceeds the maximum force it gives way, thus never allowing the force on the patient's face 18 to exceed the maximum force. The force limitation unit 26 may comprise the friction coupling 34, but may also comprise any other means that features the coupling properties of the self-releasing force-fitted coupling.

As an option, the maximum force of the force limitation unit 26 may be adjustable by applying the setting means 38, and may be different for different portions of the patient's face 18 to allow for differing sensitivity of the skin. This arrangement is particularly useful to accommodate varying external forces on the patient interface device 10 that may be induced involuntarily.

In a further alternative embodiment, the attachment assembly 14 can additionally comprise the forehead support 22 provided in the embodiment illustrated in FIG. 1. Furthermore, the headgear straps 20 can also comprise the force limitation unit 26 provided in the embodiment illustrated in FIG. 2. Hence, more than one force limitation unit 26 may be applied in the patient interface device 10. If each of the force limitation units 26 comprises setting means 38 for configuring different maximum forces, a very flexible adjustment mechanism is achieved.

Advantageously, the force limitation unit 26 can be applied regardless whether the force between the patient interface device 10 and the patient's face 18 is provided by the patient 16 or by any other means such as motors, air pressure and the like.

Furthermore, the force limitation unit 26 may also be employed not only during the fixation of the patient interface device 10 on the patient's face 18, but also during use. If the force limitation unit 26 provides an automatic adjustment of the force between the patient interface device 10 and the patient's face 18, the force limitation unit 26 may also be applied when forces are exerted on a patient interface device 10 by the patient 16 sleeping on his or her side or front.

Even if the proposed adjustment mechanism featuring the force limitation unit 26 has been described using the example of a patient interface device used for medical treatments, it shall be understood that the proposed adjustment mechanism can also be applied to any other type of mask worn by a person, such as a gas mask worn by firefighter or a soldier.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. A patient interface device comprising:
a sealing assembly for delivering a flow of breathable gas to a patient,
an attachment assembly for attaching the sealing assembly to a patient's face and
a force limitation unit for limiting a force between the patient interface device and the patient's face to a maximum force when the patient interface device is applied to the patient, the force limitation unit comprising a first and a second coupling element constituting a self-releasing force-fitted coupling for transmitting the force from the first to the second coupling element and for interrupting the force-fit between said two coupling elements when the force exceeds the maximum force.

2. The patient interface device according to claim 1, wherein the self-releasing force-fitted coupling is adapted to interrupt the force-fit between said two coupling elements by initiating a slip between said two coupling elements when the force exceeds the maximum force.

3. The patient interface device according to claim 1, wherein the self-releasing force-fitted coupling is adapted to interrupt the force-fit between said two coupling elements by at least partly releasing the linking between said two coupling elements when the force exceeds the maximum force.

4. The patient interface device according to claim 1, wherein the self-releasing force-fitted coupling is adapted to interrupt the force-fit between said two coupling elements by completely releasing the linking between said two coupling elements when the force exceeds the maximum force.

5. The patient interface device according to claim 1, wherein the attachment assembly comprises a headgear component coupled to the sealing assembly and comprising the force limitation unit.

6. The patient interface device according to claim 1, wherein the attachment assembly comprises a forehead support coupled to the sealing assembly and comprising the force limitation unit.

7. The patient interface device according to claim 1, wherein the sealing assembly comprises a rigid mask shell and a cushion coupled to the mask shell, the force limitation unit being mechanically coupled between the mask shell and the cushion.

8. The patient interface device according to claim 1, wherein the force limitation unit comprises setting means for adjusting the maximum force.

9. The patient interface device according to claim 8, further comprising a sensor for measuring the force exerted by the patient interface device on the patient's face and/or an actuator or motor for attaching the patient interface device to the patient's face, the setting means being configured to adjust the maximum force based on an output signal of the sensor, actuator and/or motor.

10. The patient interface device according to claim 1, further comprising a plurality of force limitation units being arranged on different locations of the patient interface device.

11. The patient interface device according to claim 1, wherein the force limitation unit comprises a friction coupling, a magnetic coupling, a slip coupling, a hydraulic coupling and/or an electromagnetic coupling.

12. (canceled)

13. A headgear assembly for attaching a patient interface to a patient's face, the headgear assembly comprising a force limitation unit for limiting a force between the patient interface device and the patient's face to a maximum force when the patient interface device is applied to the patient, the force limitation unit comprising a first and a second coupling element constituting a self-releasing force-fitted coupling for transmitting the force from the first to the second coupling element and for interrupting the force-fit between said two coupling elements when the force exceeds the maximum force.
